(19) United States
(12) Patent Application Publication
(10) Pub. No.: US 2025/0295725 A1
(43) Pub. Date: Sep. 25, 2025

(54) BONE TARGETED TREATMENT IN OSTEOGENESIS IMPERFECTA

(71) Applicant: PURDUE RESEARCH FOUNDATION, West Lafayette, IN (US)

(72) Inventors: Stewart Andrew LOW, West Lafayette, IN (US); Jeffery NIELSEN

(21) Appl. No.: 18/728,874

(22) PCT Filed: Sep. 13, 2022
(86) PCT No.: PCT/US2022/043358

§ 371 Date: Jul. 13, 2024

Related U.S. Application Data
(60) Provisional application No. 63/299,746, filed on Jan. 14, 2022.

(51) Int. Cl. A61K 38/04; A61K 31/381; A61K 31/65; A61K 31/662; A61K 33/42; A61K 38/03; A61P 19/08

(52) U.S. Cl. CPC A61K 38/04; A61K 31/381; A61K 31/65; A61K 31/662; A61K 33/42; A61K 38/03; A61P 19/08

(57) ABSTRACT

Disclosed herein are compounds and methods for treating Osteogenesis Imperfecta in an individual. In some embodiments, the compounds include a bone anabolic agent, a linker, and a bone targeting ligand. In some embodiments, the methods include reducing the incidences of or likelihood of bone fracture, increasing bone density or strength of a bone, and providing compounds to low density and weakened bone sites in an individual with Osteogenesis Imperfecta.

### CROSS-REFERENCE

This application claims the benefit, and is a 35 U.S.C. 371 national state application, of International Patent Application No. PCT/US22/43358 filed on Sep. 13, 2022, which claims the priority benefit of U.S. Provisional Application No. 63/299,746 filed on Jan. 14, 2022. The contents of each of the foregoing applications are hereby incorporated by reference in their entireties into this disclosure.

### BACKGROUND

Osteogenesis imperfecta (OI), also known as brittle bone disease, is a genetic disorder affecting approximately 1 in 15,000 births. Phenotypes range from occasional bone fractures due to mild trauma to severe skeletal deformities and extremely fragile bones. Individuals with OI can have upwards of 400 fractures in their lifetime, and repair can be significantly delayed. Treatment of many OI fractures differ little from traditional fracture therapy and rely primarily on stabilization. Some effort to increase the repair process in individuals with OI with drugs, such as teriparatide, has yielded only mild improvements, potentially due to the insufficient concentrations at fracture sites. Therefore, there exists a need for improved fracture-targeted therapeutics with high affinity to bone fractures sites and sites of low density and weakened bone to help treat and reduce the incidences of or likelihood of bone fractures in individuals suffering from OI.

### SUMMARY

Provided in certain embodiments herein is a method of treating Osteogenesis Imperfecta (OI) in an individual (e.g., in need thereof), the method comprising administering to the individual (e.g., in need thereof) a therapeutically effective amount of a compound, or a pharmaceutically acceptable salt thereof, having a structure XYZ, in which X is a bone anabolic agent, Y is a linker, and Z is a bone targeting ligand.

Provided in certain embodiments herein is a method of delivering (e.g., targeting) a compound described herein, or a pharmaceutically acceptable salt thereof, to one or more damaged, low density, or weakened bone sites (e.g., bone fractures, areas outside of a fracture site, such as a skeletal remodeling site or a low collagen site, or in the absence of a fracture site), such as in an individual having Osteogenesis Imperfecta (OI). In some embodiments, the one or more damaged, low density, or weakened bone sites is an osteotomy, a bone graft, a bone fracture (e.g., a microfracture or a stress fracture), or a skeletal remodeling area. In some embodiments, the method includes administering the compound to the individual, wherein the compound has a structure XYZ, wherein X is a bone anabolic agent, Y is a linker, and Z is a bone targeting ligand. In some embodiments, a therapeutically effective amount of a compound provided herein is administered and delivered to one or more damaged, low density, or weakened bone sites (e.g., bone fractures, areas outside of a fracture site, such as a skeletal remodeling site or a low collagen site, or in the absence of a fracture site). In some embodiments provided herein, administering the compound delivers (e.g., targets) a therapeutically effective amount of the compound to the one or more damaged, low density, or weakened bone sites (e.g., bone fractures, areas outside of a fracture site, such as a skeletal remodeling site or a low collagen site, or in the absence of a fracture site). In some embodiments, such as when a compound provided herein is administered as a diagnostic payload, the compound provided herein is used to identify one or more damaged, low density, or weakened bone sites (e.g., bone fractures, areas outside of a fracture site, such as a skeletal remodeling site or a low collagen site, or in the absence of a fracture site) in the individual. In some embodiments provided herein, the compound includes a diagnostic payload, and wherein administering the compound identifies the one or more damaged, low density, or weakened bone sites (e.g., bone fractures, areas outside of a fracture site, such as a skeletal remodeling site or a low collagen site, or in the absence of a fracture site) in the individual.

In some instances, the individual does not have a bone fracture.

In some embodiments provided herein, administering the therapeutically effective amount of the compound, or the pharmaceutically acceptable salt thereof, to the individual (e.g., in need thereof) reduces the incidences of or likelihood of bone fracture in the individual (e.g., in need thereof). In some embodiments, the therapeutically effective amount of the compound, or the pharmaceutically acceptable salt thereof, increases density and/or strength (e.g., mechanical, or structural strength) of a bone of the individual (e.g., in need thereof, such as an individual having OI). In some embodiments provided herein, administering the therapeutically effective amount of the compound, or the pharmaceutically acceptable salt thereof, to the individual (e.g., in need thereof) increases bone density or strength (e.g., mechanical, or structural strength) of a bone in the individual. In some embodiments, the therapeutically effective amount of the compound, or the pharmaceutically acceptable salt thereof, improves (e.g., accelerates) treatment (e.g., healing or repair) of a bone (e.g., an osteotomy, a bone graft, or a bone fracture) in the individual (e.g., in need thereof, such as an individual having OI). In some embodiments, the therapeutically effective amount of the compound, or the pharmaceutically acceptable salt thereof, reduces non-union or delayed unions of a bone (e.g., an osteotomy, a bone graft, or a bone fracture) in the individual (e.g., in need thereof, such as an individual having OI). In some embodiments, the therapeutically effective amount of the compound, or the pharmaceutically acceptable salt thereof, increases strength of a bone in the individual (e.g., in need thereof, such as an individual having OI). In some embodiments provided herein, administering the therapeutically effective amount of the compound, or the pharmaceutically acceptable salt thereof, to the individual (e.g., in need thereof) improves (e.g., accelerates) the treatment (e.g., healing or repair) of an osteotomy, a bone graft, or a bone fracture in the individual.

In some embodiments, the individual has been diagnosed with Type 1 OI. In some embodiments, the individual has been diagnosed with Type 3 OI. In some embodiments, the individual has been diagnosed with Type 4 OI. In some embodiments, the individual has been diagnosed with OI that is associated with a genetic mutation (e.g., in the individual), such as a genetic mutation that reduces collagen formation and/or production.

In some embodiments provided herein, the OI is Type 1 OI. In some embodiments provided herein, the OI is Type 3 OI. In some embodiments provided herein, the OI is Type 4 OI. In some embodiments provided herein, the OI is related to a genetic mutation in the individual in the ability to form or process collagen.

In some embodiments, the individual is an infant, a child, an adolescent, or a young adult.

In some embodiments, the individual has been diagnosed with pediatric OI.

In some embodiments provided herein, the individual is a pediatric individual having pediatric OI.

In some embodiments, a therapeutically effective amount of the compound, or the pharmaceutically acceptable salt thereof, is delivered to one or more low density and/or weakened bone sites (e.g., skeletal remodeling sites, or low collagen sites outside of a fracture site, or in the absence of a fracture site) in the individual (e.g., in need thereof). In some embodiments provided herein, the therapeutically effective amount of the compound, or the pharmaceutically acceptable salt thereof, provides a therapeutically effective amount of the compound, or the pharmaceutically acceptable salt thereof, to one or more low density or weakened bone sites (e.g., skeletal remodeling sites, or low collagen sites outside of a fracture site, or in the absence of a fracture site) in the individual (e.g., in need thereof).

In some embodiments, a compound, or a pharmaceutically acceptable salt thereof, provided herein is administered in an amount sufficient to prevent a bone fracture in an individual (e.g., in need thereof), such as an individual having OI. In some embodiments, a compound, or a pharmaceutically acceptable salt thereof, provided herein is administered in an amount sufficient to reduce the incidence of bone fracture in an individual (e.g., in need thereof), such as an individual having OI.

In some embodiments, a compound, or a pharmaceutically acceptable salt thereof, provided herein is administered prophylactically to an individual (e.g., in need thereof), such as an individual having OI. In some embodiments provided herein, methods comprised prophylactically administering to the individual (e.g., in need thereof) a therapeutically effective amount of the compound, or the pharmaceutically acceptable salt thereof.

In some embodiments, a compound, or a pharmaceutically acceptable salt thereof, provided herein is administered systemically to an individual (e.g., in need thereof), such as an individual having OI. (e.g., in need thereof), such as an individual having OI, accumulates locally (e.g., at a fracture site).

In some embodiments, a compound, or a pharmaceutically acceptable salt thereof, provided herein is administered to an individual (e.g., in need thereof), such as an individual having OI, a first time and a second time. In some embodiments, a compound, or a pharmaceutically acceptable salt thereof, provided herein is administered to an individual (e.g., in need thereof), such as an individual having OI, one or more times after the second time. In some embodiments, a compound, or a pharmaceutically acceptable salt thereof, provided herein is (repeatedly) administered to an individual (e.g., in need thereof), such as an individual having OI, for a period of a week or more. In some embodiments, a compound, or a pharmaceutically acceptable salt thereof, provided herein is (repeatedly) administered to an individual (e.g., in need thereof), such as an individual having OI, for a period of a month or more. In some embodiments, a compound, or a pharmaceutically acceptable salt thereof, provided herein is (repeatedly) administered to an individual (e.g., in need thereof), such as an individual having OI, for a period of a year or more. In some embodiments, a compound, or a pharmaceutically acceptable salt thereof, provided herein is (repeatedly) administered to an individual (e.g., in need thereof), such as an individual having OI, for several years. In some embodiments, a compound, or a pharmaceutically acceptable salt thereof, provided herein is (repeatedly) chronically administered to an individual (e.g., in need thereof), such as an individual having OI.

In some embodiments, X is a bone anabolic agent (e.g., an agent having bone anabolic activity). In some embodiments, X is a bone anabolic agent that increases the production of collagen in the individual or improves the ability of the individual to form or process collagen. In some embodiments, X is a growth factor, a small molecule, a peptide, a protein, a hormone, or a fragment thereof, such as when attached to or released from a compound described herein. In some embodiments, X is a growth factor, a small molecule, a peptide, a protein, a hormone, or a fragment thereof, such as when attached to or released from a compound described herein. In some embodiments, X is a bone anabolic agent selected from the group consisting of an agonist of parathyroid hormone receptor 1, a parathyroid hormone (PTH), a PTH-related protein (PTHrP), and abaloparatide. In some embodiments, X is Ln2P3.

In some embodiments provided herein, X is a bone anabolic agent (e.g., having bone anabolic activity) including growth factors, small molecules, peptides, proteins, or hormones. In some embodiments provided herein, X is a bone anabolic agent selected from the group consisting of an agonist of parathyroid hormone receptor 1, a parathyroid hormone (PTH) (e.g., or a derivative or fragment thereof (e.g., having bone anabolic activity)), a PTH-related protein (PTHrP) (e.g., or a derivative or fragment thereof (e.g., having bone anabolic activity)), and abaloparatide (e.g., or a derivative or fragment thereof (e.g., having bone anabolic activity)). In some embodiments provided herein, X is abaloparatide (e.g., or a derivative or fragment thereof (e.g., having bone anabolic activity)). In some embodiments, X is Ln2P3.

In some embodiments, X is an agonist of parathyroid hormone receptor 1. In some embodiments, X is a PTH. In some embodiments, X is a PTHrP. In some embodiments, X is abaloparatide. In some embodiments, X is Ln2P3.

In some embodiments provided herein, Z is a hydroxyapatite targeting ligand. In some embodiments provided herein, the hydroxyapatite targeting ligand includes a tetracycline, a phosphonate (e.g., a bisphosphonate (e.g., a mono-bisphosphonate, a tri-bisphosphonate, or a polybisphosphonate)), an acidic oligopeptide, a ranelate, a pyrophosphate, or a targeting ligand developed through phage display. In some embodiments, the hydroxyapatite targeting ligand includes a tetracycline, a phosphonate (e.g., a bisphosphonate (e.g., a mono-bisphosphonate, a tribisphosphonate, or a poly-bisphosphonate)), an acidic oligopeptide, a ranelate, and/or a pyrophosphate. In some embodiments, Z comprises a phosphonate or a derivative thereof. In some embodiments, Z comprises a phosphate or a derivative thereof. In some embodiments, Z includes one or more amino acid residue. In some embodiments provided herein, Z is a linear chain of amino acid residues. In some embodiments provided herein, Z is a branched chain of amino acid residues. In some embodiments provided herein, Z is an acidic oligopeptide. In some embodiments provided herein, Z includes at least 4 glutamic acid amino acid residues or at least 4 aspartic acid amino acid residues. In some embodiments provided herein, Z includes at least 4 (e.g., acidic) amino acid residues (e.g., having the same chirality). In some of the embodiments provided herein, each of the at least 4 (e.g., acidic) amino acid residues has D chirality. In some of the embodiments provided herein, Z includes at least 4 (e.g., D-) glutamic acid amino acid residues (e.g., 4 to 20 D-glutamic acid amino acid residues) and/or at least 4 (e.g., D-) aspartic acid amino acid residues (e.g., 4 to 20 D-aspartic acid amino acid residues). In some of the embodiments provided herein, Z comprises a mixture of (e.g., D-) glutamic acid amino acid residues and (e.g., D-) aspartic acid amino acid residues. In some of the embodiments provided herein, Z includes at least 10 repeating D-glutamic acid amino acid residues (e.g., DE10 or more, DE15 or more, or DE20 or more). In some of the embodiments provided herein, Z is 20 repeating D-glutamic acid amino acid residues (DE20). In some of the embodiments provided herein, Z includes 4 to 75 acidic amino acid residues (e.g., D-glutamic acid amino acid residues). In some of the embodiments provided herein, Z includes 8 to 30 acidic amino acid residues (e.g., D-glutamic acid amino acid residues and/or D-aspartic acid amino acid residues). In some of the embodiments provided herein, Z includes 8 to 30 D-glutamic acid amino acid residues. In some embodiments, Z includes 8 to 30 D-aspartic acid amino acid residues.

In some embodiments provided here, X is abaloparatide (e.g., or a derivative or fragment thereof (e.g., having bone anabolic activity)) and Z is 20 repeating D-glutamic acid amino acid residues (DE20).

In some of the embodiments provided herein, Y is a non-releasable linker (e.g., containing at least one carbon-carbon bond and/or at least one amide bond).

In some of the embodiments provided herein, Y is a releasable linker (e.g., containing at least one disulfide (SS), at least one ester (e.g., O(C=O)), and/or at least one (e.g., protease-specific) amide bond.

In some of the embodiments provided herein, X is abaloparatide (e.g., or a derivative or fragment thereof (e.g., having bone anabolic activity)), Y is a non-releasable oligopeptide linker, and Z is 20 repeating D-glutamic acid amino acid residues (DE20).

In some of the embodiments provided herein, X is abaloparatide (e.g., or a derivative or fragment thereof (e.g., having bone anabolic activity)), Y is a releasable oligopeptide linker comprising at least one protease-specific amide bond, and Z is 20 repeating D-glutamic acid amino acid residues (DE20).

In some of the embodiments provided herein, the compound has at least 75% sequence identity or more (e.g., at least 80% sequence identify or more, at least 85% sequence identify or more, at least 90% sequence identify or more, or at least 95% sequence identify or more) to SEQ ID NO.: 1.

In some embodiments, a compound described herein, such as a compound for treating Osteogenesis Imperfecta (OI) in an individual (e.g., in need thereof), is Compound 1.

In some of the embodiments, X is PTHrP, Y is a non-releasable oligopeptide linker, and Z is 20 repeating D-glutamic acid amino acid residues (DE20).

In some of the embodiments, the compound has at least 75% sequence identity or more (e.g., at least 80% sequence identify or more, at least 85% sequence identify or more, at least 90% sequence identify or more, or at least 95% sequence identify or more) to SEQ ID NO.: 3.

In some embodiments, a compound described herein, such as a compound for treating Osteogenesis Imperfecta (OI) in an individual (e.g., in need thereof), is Compound 3.

In some of the embodiments, X is Ln2P3, Y is a non-releasable oligopeptide linker, and Z is 10 repeating D-glutamic acid amino acid residues (DE 10).

In some of the embodiments, the compound has at least 75% sequence identity or more (e.g., at least 80% sequence identify or more, at least 85% sequence identify or more, at least 90% sequence identify or more, or at least 95% sequence identify or more) to SEQ ID NO.: 4.

In some embodiments, a compound described herein, such as a compound for treating Osteogenesis Imperfecta (OI) in an individual (e.g., in need thereof), is Compound 4.

### BRIEF DESCRIPTION OF THE SEQUENCE LISTINGS

The written Sequence Listings for the sequences described herein are the same sequence listings are provided in computer readable form in a Sequence Listing XML file (file entitled “70671-03_SequenceListing_f17feb.2025”; file size=8,450 bytes; date created Feb. 17, 2025) filed herewith and incorporated herein by reference. The information recorded in computer readable form is identical to the written sequences provided herein, pursuant to 37 C.F.R. § 1.821(f).

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows the impact of treatment on bone fracture callus mineralization using compounds disclosed herein.

FIG. 2 shows the impact of treatment on bone fracture strength using compounds disclosed herein.

FIG. 3 shows the impact of treatment on bone fracture strength using compounds disclosed herein.

FIG. 4 shows the impact of treatment on bone fracture callus mineralization using compounds disclosed herein.

FIG. 5 shows the impact of treatment on bone fracture strength using compounds disclosed herein.

FIG. 6 shows microCT images from the fractured bones in FIGS. 4 and 5.

FIG. 7 shows the impact of treatment on bone fracture strength using compounds disclosed herein.

FIG. 8 shows the impact of treatment on bone fracture strength using compounds disclosed herein.

FIG. 9 shows the impact of treatment on bone fracture strength in males using compounds disclosed herein.

FIG. 10 shows the impact of treatment on bone strength in females using compounds disclosed herein.

FIG. 11 shows the impact of treatment on bone strength using compounds disclosed herein.

FIG. 12 shows the impact of treatment on bone fracture strength using compounds disclosed herein.

FIG. 13 shows the impact of treatment on bone fracture stiffness using compounds disclosed herein.

FIG. 14 shows the impact of treatment on bone fracture stiffness using compounds disclosed herein.

FIG. 15A shows a SPEC/CT image of the Tc chelator EC20 chelating m99Tc linked to compounds disclosed herein imaged 21 hours post injection. FIG. 15B, which can be superimposed with FIG. 15C, shows the skeleton of the SPEC/CT imaged mouse. FIG. 15C, which can be superimposed with FIG. 15B, shows the localization of Compound 2 in the SPEC/CT imaged mouse.

FIG. 16 shows a SPEC/CT image of the Tc chelator EC20 chelating m99Tc linked to compounds disclosed herein imaged 21 hours post injection.

FIG. 17 shows the impact of treatment on bone volume using compounds disclosed herein.

FIG. 18 shows the impact of treatment on bone fracture strength using compounds disclosed herein.

FIG. 19 shows the impact of treatment on bone fracture strength using compounds disclosed herein.

FIG. 20 shows the impact of treatment on work to fracture using compounds disclosed herein.

FIG. 21 shows the impact of treatment on bone fracture strength using compounds disclosed herein.

FIG. 22 shows the impact of treatment on work to fracture using compounds disclosed herein.

FIG. 23 shows the impact of treatment on bone fracture callus mineralization using compounds disclosed herein.

### DETAILED DESCRIPTION

Provided in some embodiments herein is a compound for treating fractured and/or weakened (e.g., low density, brittle) bones in an individual (e.g., in need thereof). In some embodiments, the individual in need thereof has Osteogenesis Imperfecta (OI). In some embodiments, the individual has been diagnosed with Type 1 OI, Type 3 OI, and/or Type 4 OI. In some embodiments, the individual has been diagnosed with OI that is associated with a genetic mutation (e.g., in the individual), such as a genetic mutation that reduces collagen formation and/or production. In some embodiments, the individual has been diagnosed with pediatric OI.

Provided in certain embodiments herein are compounds and methods of treating fractured and/or weakened (e.g., low density, brittle) bones in an individual (e.g., in need thereof) having one or more bone diseases. Provided in certain embodiments herein are compounds and methods of treating Osteogenesis Imperfecta (OI) in individuals (e.g., in need thereof). In some embodiments, compounds described herein are administered to an individual (e.g., in need thereof) to treat the OI. In some embodiments, administering the compounds to the individual (e.g., in need thereof) provides significant improvements to bone conditions related to the OI. In some embodiments, the compounds described herein are targeted compounds. In some embodiments, the targeted compounds have targeted specificity to fracture, low density, and/or weakened bone sites.

In some embodiments, the individual does not have a bone fracture.

In some embodiments, administering the compounds described herein improves and accelerates the repair of a bone fracture in an individual in the OI disease state.

In some embodiments, a compound described herein accelerates the repair of a bone fracture in an individual (e.g., in the OI disease state) without ectopic bone formation.

In some instances, individuals with OI have numerous sites of skeletal remodeling (e.g., bone turnover), such as a result of the poor skeletal phenotype disrupting normal bone homeostasis. In some instances, the increased skeletal remodeling (e.g., bone turnover) in individuals with OI leads to numerous weakened and low density bone sites throughout the individual in varying degrees. As such, it would be surprising if increasing skeletal remodeling (e.g., bone turnover (e.g., such as by administering a compound as described herein)) improved bone healing (e.g., bone fracture healing) in individuals with OI.

In some instances, individuals with OI have a genetic mutation that reduces collagen formation and/or production, such as leading to poor collagen synthesis. In some instances, poor collagen synthesis is associated with low amounts of hydroxyapatite in bones. As such, it would be surprising if a compound (e.g., a bone anabolic compound) that targets hydroxyapatite significantly increased bone healing rates and/or improved the bone strength (e.g., mechanical and structural bone strength) of an individual diagnosed with OI. Yet, provided in some embodiments herein a compound as described herein, or a pharmaceutically acceptable salt thereof, improves (e.g., accelerates) the treatment (e.g., healing or repair) of a bone (e.g., an osteotomy, a bone graft, or a bone fracture) in an individual with OI. In some embodiments, administering the compounds described herein improves callus mineralization in individuals with OI.

In some embodiments, the therapeutically effective amount of the compound, or the pharmaceutically acceptable salt thereof, reduces non-union or delayed unions of a bone (e.g., an osteotomy, a bone graft, or a bone fracture) in the individual (e.g., in need thereof, such as an individual having OI).

In some embodiments, the therapeutically effective amount of the compound, or the pharmaceutically acceptable salt thereof, increases strength of a bone in the individual (e.g., in need thereof, such as an individual having OI).

In some instances, ability of an anabolic linked to a hydroxyapatite ligand to accelerate the repair of fractures in a Type 3 OI disease state was analyzed. Type 3 is the most severe non-lethal form of OI and experiences the most fractures in the human population. In some instances, Type 3 was modeled in a Colla2oim (−/−) mouse (e.g., which is a well-established model of OI Type 3), such as described elsewhere herein (e.g., see the examples). In some instances, the compound is distally administered and accumulates locally at a fracture site.

In some instances, a marked increase in bone volume fraction (e.g., >85%) was observed in mice administered a compound provided herein (e.g., Ab46-D-Glu20) over the saline groups. In some instances, mechanical testing yielded between 220% and 300% increase in force to fracture in in mice administered a compound provided herein (e.g., Ab46-D-Glu20) over the saline control groups.

In some instances, a significant improvement of radiographic healing was observed with administration of a compound provided herein (e.g., Ab46-D-Glu20) over the control. In some instances, the Colla2oim mouse model (e.g., as with most OI cases) causes a defect in collagen formation, such as leading to poor bone quality. While radiographic healing may not fully elucidate that the overall mechanical quality of bone may still be impaired, mechanical testing elucidates the quality of the bone. In some instances, a dramatically higher force was required to refracture bone after a compound provided herein (e.g., Ab46-D-Glu20) was administered. In some instances, the improvement in mechanical stability (e.g., in the context of OI) would mean that once a fracture is healed radiographically (and an individual has their stabilizing cast or splint removed) that an individual would have sufficient bone strength to return to normal activity without fear of refracture. In some instances, targeted bone anabolics provided herein (e.g., Ab46-D-Glu20) improve the structural and mechanical healing of bone fractures in both adult and pediatric type 1 and 3 OI. In some instances, bone targeted therapeutics provided herein (e.g., Ab46-D-Glu20) improve and accelerate fracture healing as well as prevent future bone fractures, such as improving the overall quality of life for OI patients. OI is an underserved population that would greatly benefit from a treatment that, in conjunction with conventional therapy, not only improves fracture repair but is safe enough to use several times throughout their lives. By targeting bone anabolic agents to bone fractures, sufficient concentrations of anabolic agent can be delivered to the fracture site to safely accelerate healing. In some instances, such as when a physician observes radiographic healing, an individual is much more likely to be cleared to function without a cast splint (and to continue with normal activities). In some instances, such as in the process of measuring bone volume fraction, the measurement to the bridging volume of the callus was limited, such as to focus measurements on radiographic healing.

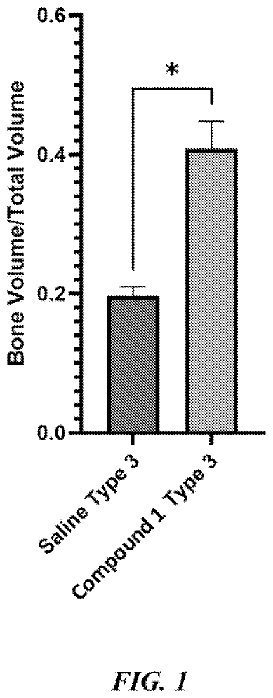

FIG. 1 shows the impact of treatment with Compound 1 (SEQ ID NO.: 1, AVSEHQLLHDKGKSIQDLRRRELLEKLLxKLHTAEIRATSEVSPNSeeeeeeeeeeeeeeeeeeee, where x=Aib and “e” signifies D-glutamic acid), on bone fracture callus mineralization in a Col 1a2^{0im} mouse model of type 3(−/−) OI. After reaching skeletal maturity, 12 weeks old female mice underwent a stabilized midshaft femur fracture model via an osteotomy. Type 3 OI mice were treated twice a week 6 weeks post fracture. Fracture callus mineralization was evaluated via quantification of high resolution MicroCT(scanco) images of the fracture callus. Compound 1 significantly improved the fracture callus mineralization for Type 3 OI mice. As shown in FIG. 1, Compound 1 provides improved fracture callus mineralization by approximately 100% as compared to the saline control. In some instances, improved callus mineralization provides structurally improved bone in individuals with OI.

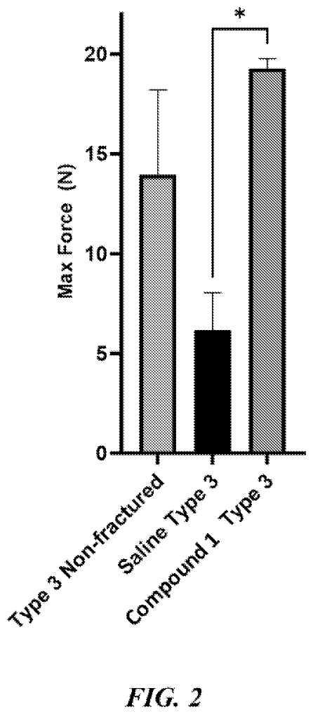

In some instances, compounds described herein provide mechanically improved bone in individuals with OI. FIG. 2 shows the impact of treatment with Compound 1 on bone fracture strength in a Colla2^{0im} mouse model of type 3(−/−) OI. After reaching skeletal maturity, 12 weeks old female mice underwent a stabilized midshaft femur fracture model via an osteotomy. Type 3 OI mice were treated twice a week for 6 weeks post fracture. Fracture strength was evaluated via 4-point bend to failure of the fractured femurs. The max load evaluates the max load the femur withstood prior to fracture in the biomechanical evaluation. The contralateral untreated femurs are presented here to show the restoration to previous non-fracture strength during treatment. As shown in FIG. 2, Compound 1 increases the max load (in Newtons) it takes a bone to fracture by approximately 300% (as compared to saline bone). FIG. 2 also shows that Compound 1 treated bone has a higher max load requirement to fracture by approximately 40% as compared to non-fractured bone.

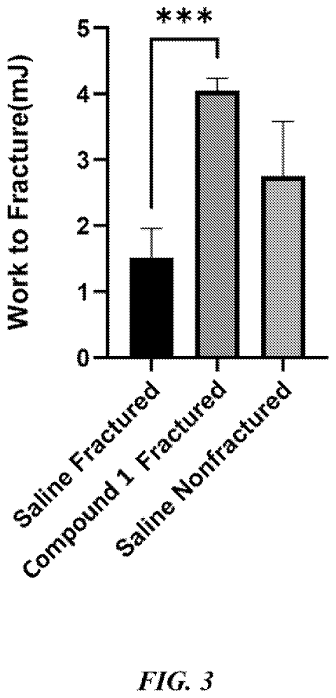

FIG. 3 shows the impact of treatment with Compound 1 on bone fracture strength in the Colla2oim mouse model of type 3(−/−) OI. After reaching skeletal maturity, 12 weeks old female mice underwent a stabilized midshaft femur fracture model via an osteotomy. Type 3 OI mice were treated twice a week for 6 weeks post fracture. Fracture strength was evaluated via 4-point bend to failure of the fractured femurs. Work to fracture evaluates the total amount of energy the femur adsorbed prior to fracture in the biomechanical evaluation. The contralateral untreated femurs are presented here to show the restoration to previous non-fracture strength during treatment. As shown in FIG. 3, the work to fracture, or total amount of energy a bone adsorbs prior to fracture, for bone treated with Compound 1 is increased by approximately 150% as compared to bone repaired with saline, and by approximately 70% as compared to non-fractured bone.

Provided in certain embodiments herein is a method for treating Osteogenesis Imperfecta (OI) in an individual (e.g., in need thereof), the method comprising administering to the individual (e.g., in need thereof) a therapeutically effective amount of a compound, or a pharmaceutically acceptable salt thereof, having a structure X-Y-Z, wherein X is a bone anabolic agent, Y is a linker, and Z is a bone targeting ligand.

In some embodiments, a compound, or a pharmaceutically acceptable salt thereof, provided herein is administered in an amount sufficient to prevent a bone fracture in an individual (e.g., in need thereof), such as an individual having OI. In some embodiments, a compound, or a pharmaceutically acceptable salt thereof, provided herein is administered in an amount sufficient to reduce the incidence of bone fracture in an individual (e.g., in need thereof), such as an individual having OI.

In some embodiments, the compound, or the pharmaceutically acceptable salt thereof, is prophylactically administered to the individual (e.g., in need thereof) in a therapeutically effective amount.

In some embodiments, the compound, or the pharmaceutically acceptable salt thereof, is systemically administered to the individual (e.g., in need thereof) in a therapeutically effective amount.

In some embodiments, the compound, or the pharmaceutically acceptable salt thereof, is distally administered to the individual (e.g., in need thereof) in a therapeutically effective amount. In some embodiments, a compound, or a pharmaceutically acceptable salt thereof, that has been distally administered to an individual (e.g., in need thereof) accumulates locally at a fracture site.

In some embodiments, a compound, or the pharmaceutically acceptable salt thereof, provided herein reduces the incidence or likelihood of bone fracture in an individual (e.g., in need thereof), such as an individual diagnosed with OI. In some embodiments, a compound, or the pharmaceutically acceptable salt thereof, provided herein increases bone density or bone strength (e.g., mechanical or structural bone strength) in the individual. In some embodiments, a therapeutically effective amount of a compound provided herein is an amount effective at reducing severity or incidence of a bone fracture, such as on average.

In some embodiments, administering the therapeutically effective amount of the compound, or the pharmaceutically acceptable salt thereof, to the individual (e.g., in need thereof) reduces the incidences of or likelihood of bone fracture in the individual (e.g., in need thereof). In some embodiments, administering the therapeutically effective amount of the compound, or the pharmaceutically acceptable salt thereof, to the individual (e.g., in need thereof) increases bone density or strength (e.g., mechanical, or structural strength) of a bone in the individual. In some embodiments, reducing the incidences of or likelihood of bone fracture in the individual (e.g., in need thereof) is accomplished by prophylactically administering the compound, or the pharmaceutically acceptable salt thereof, to the individual (e.g., in need thereof) in a therapeutically effective amount. In some embodiments, reducing the incidences of or likelihood of bone fracture in the individual (e.g., in need thereof) is accomplished by systemically administering the compound, or the pharmaceutically acceptable salt thereof, to the individual (e.g., in need thereof) in a therapeutically effective amount.

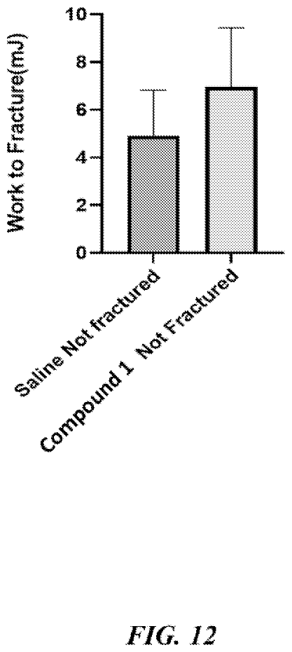

FIGS. 11 and 12 show the impact of treatment with Compound 1 on bone strength in a Colla2^{0im} mouse model of type!(+/−) OI. 12 week old female mice underwent a stabilized midshaft femur fracture model via an osteotomy. Type 1 OI mice were treated twice a week for 5 weeks post fracture. Bone strength was evaluated via 4-point bend to failure of the non-fractured contralateral femurs. The max load (FIG. 11) evaluates the max load the femur withstood prior to fracture in the biomechanical evaluation. Work to fracture (FIG. 12) evaluates the total amount of energy the femur adsorbed prior to fracture in the biomechanical evaluation. Bone targeted anabolic treatment dramatically improves bone strength in the contralateral femurs. As shown in FIG. 11, non-fractured bone in mice treated with Compound 1 take approximately 30% more max load to fracture than nonfractured bone in control mice. As shown in FIG. 12, the work to fracture, or total amount of energy the non-fractured bone can adsorb prior to fracture, is approximately 40% higher in non-fractured bone of mice treated with Compound 1 than control mice.

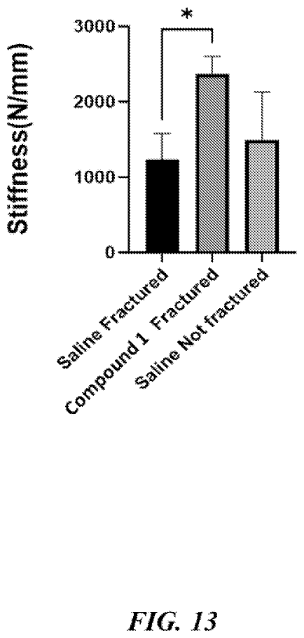

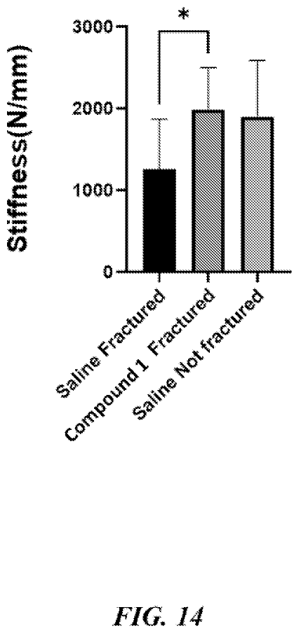

FIGS. 13 and 14 show the impact of treatment with Compound 1 on bone fracture stiffness in a Colla2^{oim} mouse model of type 3(−/−) and type 1(−/+) osteogenesis Imperfecta. After reaching skeletal maturity, 12 weeks old female mice underwent a stabilized midshaft femur fracture model via an osteotomy and type 1 and Type 3 OI mice were treated twice a week for 5 and 6 weeks respectively post fracture. Fracture strength was evaluated via 4-point bend to failure of the fractured femurs. Modulus evaluates the stiffness of the femur during the biomechanical evaluation. A low stiffness is indicative of brittleness that leads to leads to future fractures. The improvement in stiffness that occurred in the fracture callus are indicative of a healthier bone that is less brittle and will be less likely to fracture in the future. In some instances, low bone stiffness is indicative of brittleness in the bone, such as being associated with higher risk of fracture. In some instances, higher bone stiffness is indicative of healthier bone, such as bone that is less brittle and less prone to fracture. In some instances, the majority of the compound localized into the fracture callus (e.g., due to the large amounts of exposed hydroxyapatite at the fracture site and that DE20 can bind to hydroxyapatite via chelation of the calcium component). In some instances, the elevated sites of resorption throughout the OI type 3 skeleton also had some moderate accumulation (e.g., which was responsible for the non-fracture skeletal improvements observed in FIGS. 13 and 14). In some instances, such as in the type 3 mice with no fracture, an accumulation in sites of high bone turnover over throughout the skeleton were still observed, such as demonstrating this targeting technique can be used to deliver therapeutics to the sites of bone damage and high turnover in individuals with OI in the absence of fractures prophylactically to strengthen the bone and prevent fractures As shown in FIG. 13, fractured bone of mice with Type 3 OI treated with Compound 1 are approximately 100% more stiff than fractured bone of control mice, and approximately 50% more stiff than non-fractured bone of control mice. As shown in FIG. 14, fractured bone of mice with Type 1 OI treated with Compound 1 are approximately 60% more stiff than fractured bone of control mice, and approximately 10% more stiff than unfractured bone of control mice.

OI is viewed as a composite disease characterized by many subtypes that are often related to genetic mutation in the ability to form or process proper collagen. In some embodiments, a compound provided herein is effective at improving bone health in individuals with different types of OI. In some embodiments, the OI is type 4 OI. In some embodiments, the OI is related to a genetic mutation in the individual in the ability to form or process collagen.

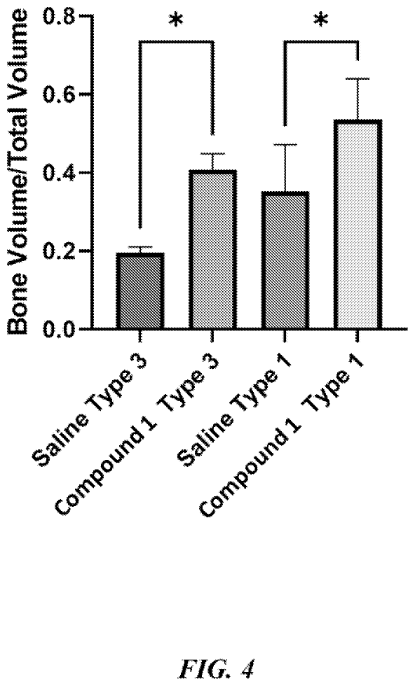

FIG. 4 shows the impact of treatment with Compound 1 on bone fracture callus mineralization in a Colla2^{oim} mouse model of type 1(+/−) and type 3(−/−) OI. After reaching skeletal maturity, 12 weeks old female mice underwent a stabilized midshaft femur fracture model via an osteotomy. Type 1 and Type 3 OI mice were treated twice a week for 5 weeks and 6 weeks respectively post fracture. Fracture callus mineralization was evaluated via quantification of high resolution MicroCT(scanco) images of the fracture callus. Compound 1 significantly improved the fracture callus mineralization for both Type 1 and Type 3 OI mice. As shown in FIG. 4, Compound 1 provides improved fracture callus mineralization by approximately 100% as compared to the saline control in mice with Type 3 OI, and by approximately 50% as compared to the saline control in mice with Type 1 OI. In some instances, improved callus mineralization provides structurally improved bone in individuals with OI.

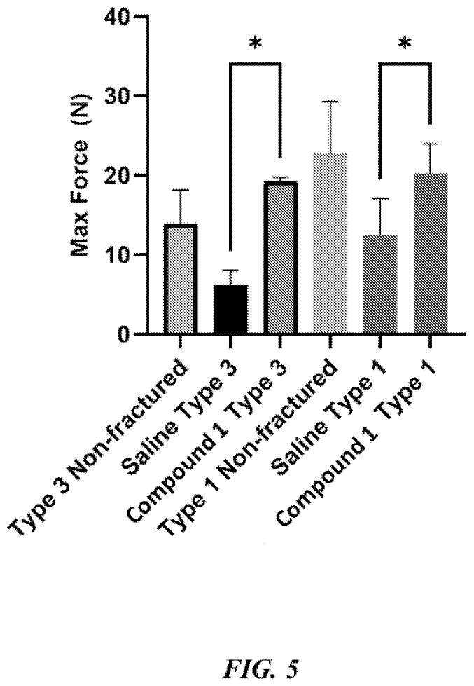

FIG. 5 shows the impact of treatment with Compound 1 on bone fracture strength in a Colla2^{oim} mouse model of type 1(+/−) and type 3(−/−) OI. After reaching skeletal maturity, 12 weeks old female mice underwent a stabilized midshaft femur fracture model via an osteotomy. Type 1 and Type 3 OI mice were treated twice a week for 5 weeks and 6 weeks respectively post fracture. Fracture strength was evaluated via 4-point bend to failure of the fractured femurs. The contralateral untreated femurs are presented here to show the restoration to previous non-fracture strength during treatment. As shown in FIG. 5, Compound 1 increases the max load (in Newtons) it takes a bone to fracture in mice with Type 3 OI by approximately 300% (as compared to bone with saline), and by about 40% as compared to Type 3 non-fractured bone. Further, Compound 1 increases the max load (in Newtons) it takes a bone to fracture in mice with Type 1 OI by approximately 70% (as compared to bone with saline).

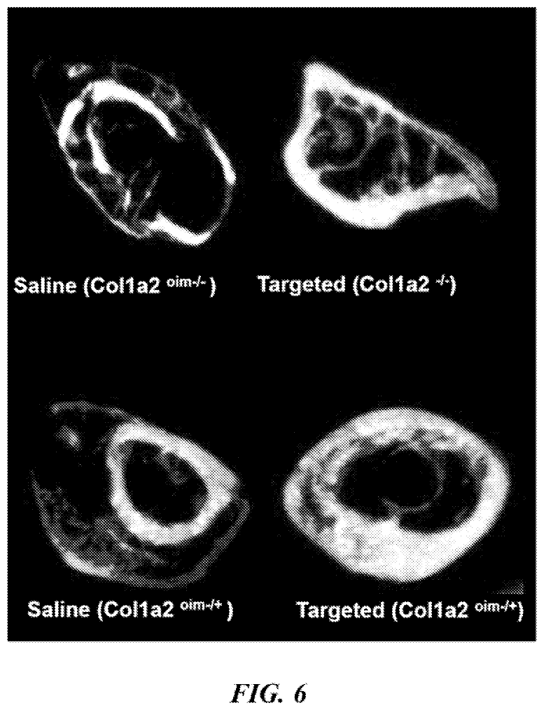

FIG. 6 shows Median microCT images from fractured bones in FIGS. 4 and 5. Each image is a composite of 20 microCT slices. Lighter shading indicates higher bone densities than darker shading. The white areas constitute original cortical bone and the darker shading correspond to new trabecular and woven bone. As shown in FIG. 6, Compound 1 facilitates healthier and denser bone (e.g., lighter shading) than the saline control.

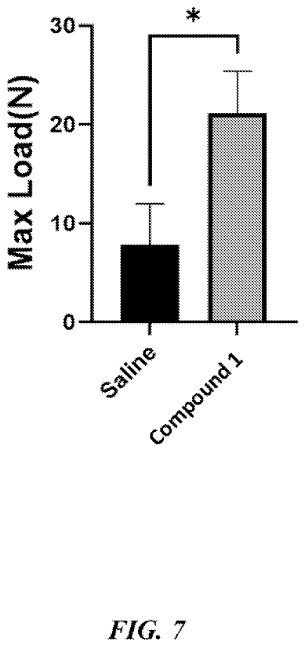

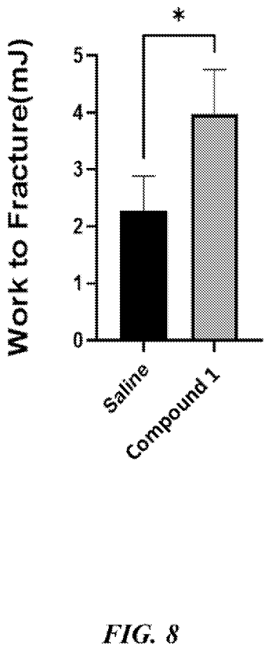

In some instances, OI affects children from birth and many of the fractures in an individual's life with OI occurring during adolescence. In some embodiments, the individual has pediatric OI. FIGS. 7 and 8 show the impact of treatment with Compound 1 on bone fracture strength in a Colla2^{oim} mouse model of type 1(+/−) OI. 6 week old pediatric Mice underwent a stabilized midshaft femur fracture model via an osteotomy. Type 1 OI mice were treated twice a week for 4 weeks post fracture. Fracture strength was evaluated via 4-point bend to failure of the fractured femurs. The max load (FIG. 7) evaluates the max load the femur withstood prior to fracture in the biomechanical evaluation. Work to fracture (FIG. 8) evaluates the total amount of energy the femur adsorbed prior to fracture in the biomechanical evaluation. As shown in FIG. 7, the max load it takes the bone to fracture in pediatric mice is approximately 160% higher with Compound 1 than bone in mice administered the saline control. As shown in FIG. 8, the work to fracture, or total amount of energy the bone in pediatric mice can adsorb prior to fracture, is approximately 80% higher in the bone of mice administered Compound 1 than the bone of mice administered saline control.

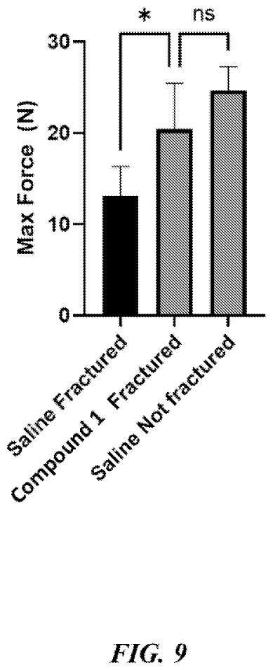

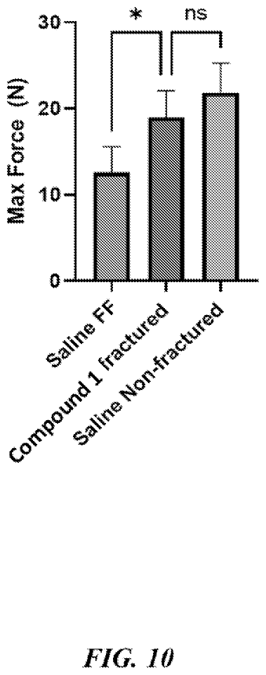

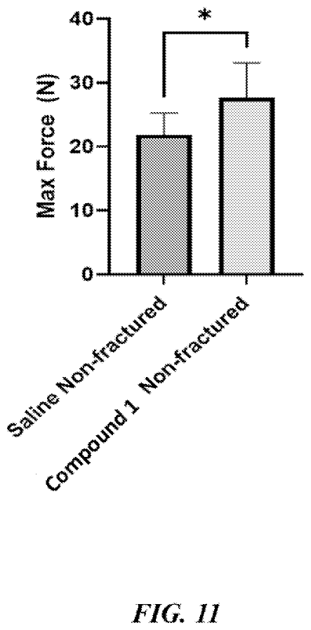

In some embodiments, the individual is a male. In some embodiments the individual is a female. In some instances, the effects of sex on the response of bone targeted anabolics in stimulated fracture healing in OI and the repair of stabilized mid-shaft femur fracture repair in type 1 OI Colla2oim (+/−) Mice was evaluated, such as using a method described herein. In some instances, the compound is distally administered and accumulates locally at a fracture site. FIGS. 9 and 10 show the impact of treatment with Compound 1 on bone fracture strength in a Col 1 a2^{oim} mouse model of type 1(+/−) OI in both male and female mice. 12 week old male (FIG. 9) and female (FIG. 10) mice underwent a stabilized midshaft femur fracture model via an osteotomy. Type 1 OI mice were treated twice a week for 5 weeks post fracture. Fracture strength was evaluated via 4-point bend to failure of the fractured femurs. The max load evaluates the max load the femur withstood prior to fracture in the biomechanical evaluation. The contralateral untreated femurs are presented to show the restoration to previous non-fracture strength during treatment. No differential response between sexes was observed. As shown in FIG. 9, fractured bone in male mice treated with Compound 1 take approximately 70% more max load to fracture than fractured bone in control mice. As shown in FIG. 10, fractured bone in female mice treated with Compound 1 take 80% more max load to fracture than fractured bone in control mice.

In some embodiments, a therapeutically effective amount of a compound provided herein is delivered to one or more low density or weakened bone sites (e.g., skeletal remodeling sites, or low collagen sites outside of a fracture site, or in the absence of a fracture site) in an individual (e.g., in need thereof), such as an individual having OI. In some embodiments, the therapeutically effective amount of the compound, or the pharmaceutically acceptable salt thereof, provides a therapeutically effective amount of the compound, or the pharmaceutically acceptable salt thereof, to one or more low density or weakened bone sites (e.g., skeletal remodeling sites, or low collagen sites outside of a fracture site, or in the absence of a fracture site) in the individual (e.g., in need thereof).

Provided in certain embodiments herein is a method of delivering (e.g., targeting) a compound, or a pharmaceutically acceptable salt thereof, to one or more damaged, low density, or weakened bone sites (e.g., bone fractures, areas outside of a fracture site, such as a skeletal remodeling site or a low collagen site, or in the absence of a fracture site) in an individual having Osteogenesis Imperfecta (OI). In some embodiments, the method includes administering the compound to the individual, wherein the compound has a structure XYZ, wherein X is a bone anabolic agent, Y is a linker, and Z is a bone targeting ligand. In some embodiments, administering the compound delivers (e.g., targets) a therapeutically effective amount of the compound to the one or more damaged, low density, or weakened bone sites (e.g., bone fractures, areas outside of a fracture site, such as a skeletal remodeling site or a low collagen site, or in the absence of a fracture site). In some embodiments, the one or more damaged, low density, or weakened bone sites is an osteotomy, a bone graft, a bone fracture (e.g., a microfracture or a stress fracture), or a skeletal remodeling area. In some embodiments, the compound comprises a diagnostic payload, and which administering the compound identifies the one or more damaged, low density, or weakened bone sites (e.g., bone fractures, areas outside of a fracture site, such as a skeletal remodeling site or a low collagen site, or in the absence of a fracture site) in the individual.

In some embodiments, low density or weakened bone sites is associated with skeletal remodeling or low collagen sites. In some embodiments, low density or weakened bone sites occur as a result of skeletal remodeling or low collagen sites. In some embodiments, the skeletal remodeling sites and/or the low collagen sites are found throughout the body of the individual. In some embodiments, the skeletal remodeling sites are found at sites in the absence of bone fractures. In some embodiments, the skeletal remodeling sites and/or the low collagen sites are found at sites found outside the sites of bone fractures. In some embodiments, the skeletal remodeling sites are found at sites found outside the sites of bone fractures. In some embodiments, individuals with OI have numerous sites of elevated skeletal remodeling that further contribute to the poor skeletal phenotype associated with OI, such as due to disruption to the normal bone homeostasis. In some embodiments, such effects lead to a further osteopenic state that results in even weaker bones, such as beyond just the bones produced as a result of a genetic disruption of collagen. In some embodiments, the increased skeletal remodeling is mosaic, such as occurring in numerous sites throughout the bones of the individual, such as in varying degrees. In some embodiments, such sights of remodeling are weak or low-density bone sites, or potential sites for future fractures. In some embodiments, increased skeletal resorption provides sites of exposed hydroxyapatite outside a fracture site alone for a bone targeted anabolic to accumulate, such as leading to advantageous accumulation throughout the rest of the skeleton in sites (e.g., weakened or low density sites (e.g., bone fractures, areas outside of a fracture site, such as a skeletal remodeling site or a low collagen site, or in the absence of a fracture site)), such as that need improved bone deposition. In some instances, such as when evaluating the rest of the skeleton in OI mice treated with bone targeted anabolics, dramatic improvements in the strength of the skeleton, such as due to these nonfracture skeletal accumulations of the targeted drug (e.g., leading to increases in bone mineral density), were identified. In some instances, this demonstrates that targeted anabolics not only can improve and accelerate fracture healing, but they can be used to prevent future fractures by improving the strength of the weakened sites of the skeleton. In some embodiments, a targeted diagnostic payload is delivered to exposed hydroxyapatite. In some embodiments, delivery of the targeted diagnostic payload to exposed hydroxyapatite is useful for identifying one or more sites of weakened or low density bone (e.g., bone fractures, areas outside of a fracture site, such as a skeletal remodeling site or a low collagen site, or in the absence of a fracture site) in the individual.

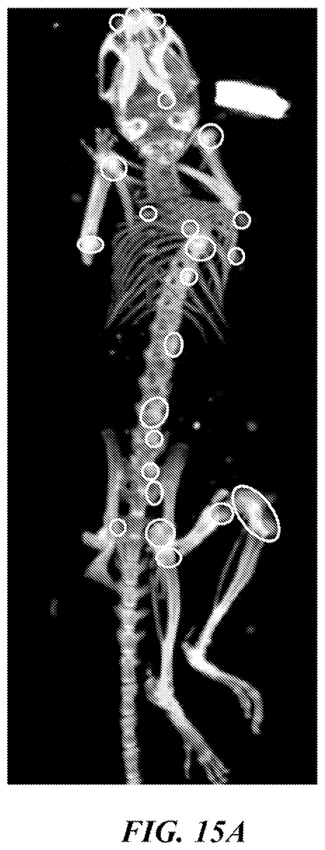

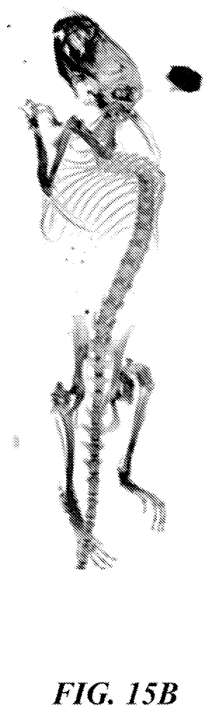

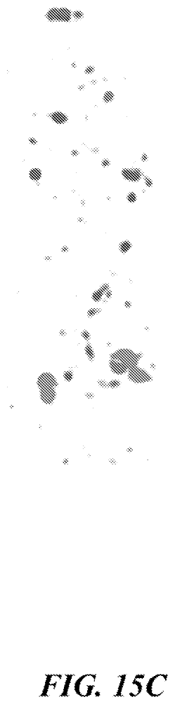

FIGS. 15A-C show imaging with Compound 1 labeled with m99Tc-EC20 (SEQ ID NO.: 2, eeeeeeeeeeeeeeeeeeee(AEEA)pDapDC((m99Tc), in which AEEA means 2,2-amino ethoxy ethoxy acetic acid, p Dap means Beta diaminoproprionic acid, ((m99Tc) means chelated m99Tc, D means aspartic acid, C means cysteine, and “e” means D-glutamic acid), such as at 21 hours using SPECT/CT (MiLabs U-SPECT-IVCT) in 12 week old female Colla2^{oim} Type 3 (−/−) mice with midshaft femur fractures 2 weeks post-fracture. As shown in FIGS. 15A-15C, Compound 2 accumulated at the site of bone fracture, as well as at sites of low density and weakened bone sites throughout the body. FIG. 15B and FIG. 15C are images of the mouse skeleton and localization of Compound 2, which can be superimposed to provide FIG. 15A.

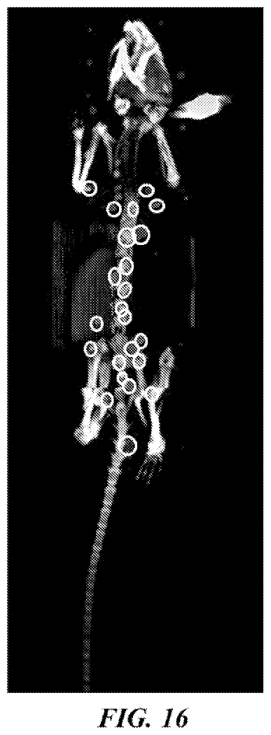

FIG. 16 shows Compound 2 and imaged at 21 hours using SPECT/CT (MiLabs U-SPECT-II/CT) in 12 week old female Colla2^{oim} Type 3 (−/−) mice without midshaft femur fractures 2 weeks post-fracture. As shown in FIG. 16, in the absence of a fracture, the Compound 2 accumulated at low density and weakened bone sites throughout the body.

In some instances, a compound provided herein increases bone volume fraction of a fractured bone in an individual in need thereof (e.g., an individual having a bone fracture and OI). In some instances, a compound provided herein significantly increases bone volume fraction of a fractured bone in an individual in need thereof (e.g., an individual having a bone fracture and OI). In some instances, a compound provided herein increases bone volume fraction of one or more fractured bone(s) in an individual in need thereof (e.g., an individual having a bone fracture and OI) by about at least 40% (e.g., about 50-55%) compared to saline control (e.g., FIG. 1).

In some instances, a compound provided herein increases force to fracture of a fractured bone in an individual in need thereof (e.g., an individual having a bone fracture and OI). In some instances, a compound provided herein significantly increases force to fracture of a fractured bone in an individual in need thereof (e.g., an individual having a bone fracture and OI). In some instances, a compound provided herein increases force to fracture of one or more fractured bone(s) in an individual in need thereof (e.g., an individual having a bone fracture and OI) by about at least 100% (e.g., 150% or more, 200% or more (e.g., by about 150-300%) compared to saline control (e.g., FIG. 2).

In some instances, a compound provided herein increases work to fracture of a fractured bone in an individual in need thereof (e.g., an individual having a bone fracture and OI). In some instances, a compound provided herein significantly increases work to fracture of a fractured bone in an individual in need thereof (e.g., an individual having a bone fracture and OI). In some instances, a compound provided herein increases work to fracture of one or more fractured bone(s) in an individual in need thereof (e.g., an individual having a bone fracture and OI) by about at least 40% (e.g., about 60%) compared to saline control (e.g., FIG. 3).

In some embodiments, a compound provided herein, such as Compound 3 (SEQ ID NO.: 3, AVSEHQLLHDKGKSIQDLIUGTFFLHHLIAEIHTAEnLATSEVSPNSeeeeeeeeeeeeeeeeeeee, where “e” stands for D-glutamic acid) localizes PTHrP to bone via targeting ligand.

Laminin is a protein component of the extracellular matrix (ECM). In some instances, laminin mediates cell attachment to surfaces either via syndecans or integrins. In some instances, the human laminin-a2 LG1 domain mediates cell attachment through syndecan-1, such as by inducing phosphorylation and membrane localization of protein 326 kinase Cd.254 Ln2_P3 or DLTIDDSYWYRI (which is one of the bioactive cores of the human laminin a2 chain.254). In some instances, laminin accelerates osteointegration of dental implants improves overall cell attachment and activates osteoblasts.

In some embodiments, a compound provided herein, such as Compound 4 (SEQ ID NO.: 4, eeeeeeeeee(AEEA)₄ DLTIDDSYWYRI, in which AEEA is 2,2-amino ethoxy ethoxy acetic acid and “e” signifies D-glutamic acid), localizes the Ln2_P3 motif to bone via an N-terminally-attached 4-mini-PEG spacer and targeting ligand.

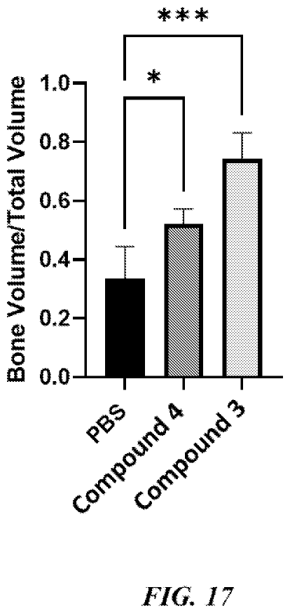

FIG. 17 shows the impact of treatment with Compound 3 and Compound 4 on bone fracture callus mineralization in a Colla2^{oim} mouse model of type 1(+/−) OI. After reaching skeletal maturity, 12 weeks old mice underwent a stabilized midshaft femur fracture model via an osteotomy. Type 1 OI mice were treated daily subcutaneously for 5 weeks post fracture. Fracture callus mineralization was evaluated via quantification of high resolution MicroCT(scanco) images of the fracture callus. In some instances, mice were dosed (daily) with 38 nmol/kg/d of Compound 4, Compound 3, or PBS vehicle control. In some instances, Compound 4 significantly improved the fracture callus mineralization for type 1 OI mice. In some instances, Compound 3 significantly improved the fracture callus mineralization for type 1 OI mice. As shown in FIG. 17, Compound 4 provides improved fracture callus mineralization by approximately 200% and Compound 3 provides improved fracture callus mineralization by approximately 300% as compared to the saline control. In some instances, improved callus mineralization provides structurally improved bone in individuals with OI.

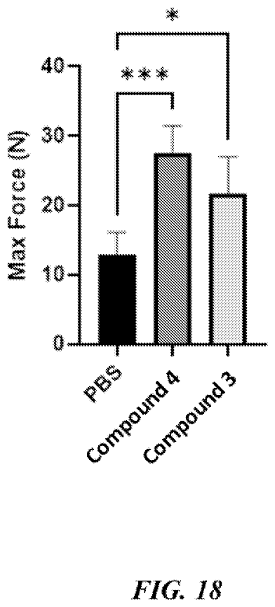

In some instances, compounds described herein provide mechanically improved bone in individuals with OI. FIG. 18 shows the impact of treatment with Compound 3 and Compound 4 on bone fracture strength in a Colla2^{0im} mouse model of type 3(−/−) OI. After reaching skeletal maturity 12 weeks old female mice underwent a stabilized midshaft femur fracture model via an osteotomy. Type 1 OI mice were treated daily subcutaneously for 5 weeks post fracture. Fracture strength was evaluated via 4-point bend to failure of the fractured femurs. In some instances, mice were dosed (daily) with 38 nmol/kg/d of Compound 4, Compound 3, or PBS vehicle control. The max load evaluates the max load the femur withstood prior to fracture in the biomechanical evaluation. The contralateral untreated femurs are presented here to show the restoration to previous non-fracture strength during treatment. As shown in FIG. 18, Compound 4 increases the max load (in Newtons) it takes a bone to fracture by approximately 200% (as compared to control). As shown in FIG. 18, Compound 3 increases the max load (in Newtons) it takes a bone to fracture by approximately 100% (as compared to control).

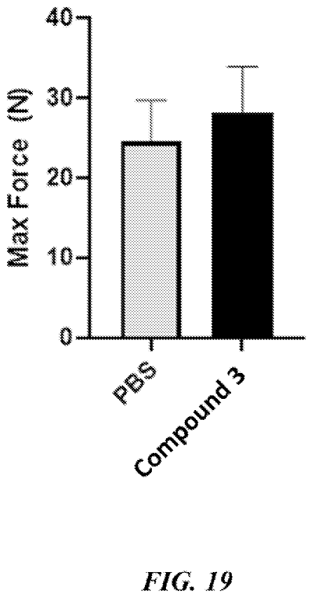

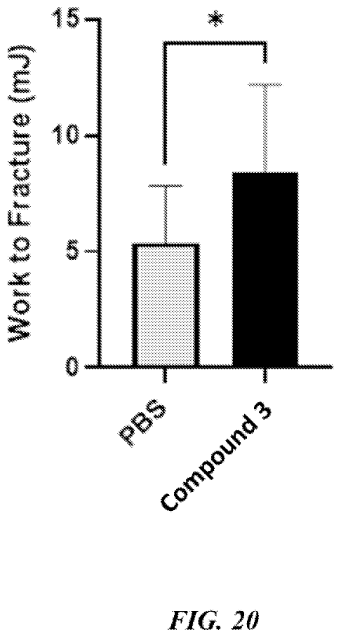

FIGS. 19 and 20 show the impact of treatment with Compound 3 on bone strength in a Colla2^{oim} mouse model of type 1(+/−) OI. 12 week old female mice underwent a stabilized midshaft femur fracture model via an osteotomy. Type 1 OI mice were treated daily subcutaneously for 5 weeks post fracture. Bone strength was evaluated via 4-point bend to failure of the non-fractured contralateral femurs. The max load (FIG. 19) evaluates the max load the femur withstood prior to fracture in the biomechanical evaluation. Work to fracture (FIG. 20) evaluates the total amount of energy the femur adsorbed prior to fracture in the biomechanical evaluation. Bone targeted anabolic treatment dramatically improves bone strength in the contralateral femurs. As shown in FIG. 19, refractured bone in mice treated with Compound 3 take approximately 10% more max load to fracture than re-fractured bone in control mice. As shown in FIG. 20, the work to fracture, or total amount of energy the non-fractured bone can adsorb prior to fracture, is approximately 40% higher in non-fractured bone of mice treated with Compound 3 than control mice.

In some instances, FIGS. 17-20 demonstrate that a targeting ligand (De10, De20, etc.) can be used to deliver therapeutic peptides beyond abaloparatide (e.g., PTHrP, Ln2P3, or the like) to bone. In some instances, Compound 4 demonstrates that de20 is not the only bone targeting ligand that can be used to localize a therapeutic peptide to a bone fracture (e.g., of mice with OI). For example, other bone targeting molecules with affinity for hydroxyapatite (e.g., other acidic peptides, phosphonates, phosphates, tetracyclines, ranelates, alendronate, or the like) could be used to localize anabolic payloads to a bone fracture in individuals with osteogenesis imperfecta.

In some instances, targeted QK effectively and significantly improves bone healing in a normal healthy state. In some instances, QK improves healing in a normal fracture by acting on endothelial cells to reestablish vascularization to the bone.

However, in the OI disease state, a main factor that delays fracture healing is the impairment in collagen, which is produced by osteoblasts. Moreover, OI is a genetic disease primarily caused by frameshift or premature stop mutations in COL1A1 or COL1A2 genes. In some instances, loss of (normal) collagen affects quality and quantity of bone (e.g., the amount of bone that forms).

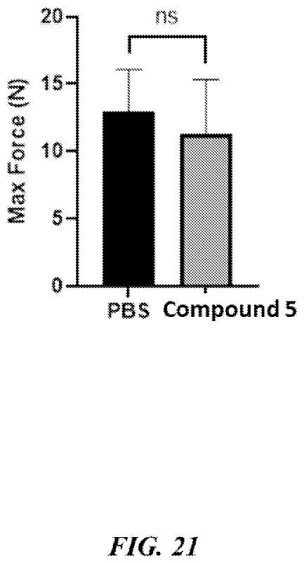

FIG. 21 shows the impact of treatment with the targeted compound QK-De10 on bone fracture strength in a Colla2^{oim} mouse model of type 1(+/−) OI. After reaching skeletal maturity 12 weeks old mice underwent a stabilized midshaft femur fracture model via an osteotomy. Type 1 OI mice were treated daily subcutaneously for 5 weeks post fracture. Fracture strength was evaluated via 4-point bend to failure of the fractured femurs. The max load evaluates the max load the femur withstood prior to fracture in the biomechanical evaluation. In some instances, mice were dosed (daily) with QK-De10 or PBS vehicle control. In some instances, QK-De10 is unable to improve the fracture callus strength for type 1 OI mice.

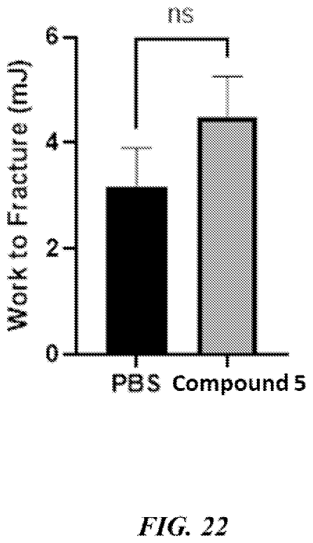

FIG. 22 shows the impact of treatment with the targeted compound QK-De10 on bone fracture strength in a Colla2^{oim} mouse model of type 1(+/−) OI. After reaching skeletal maturity 12 weeks old mice underwent a stabilized midshaft femur fracture model via an osteotomy. Type 1 OI mice were treated daily subcutaneously for 5 weeks post fracture. Fracture strength was evaluated via 4-point bend to failure of the fractured femurs. Work to fracture evaluates the total amount of energy the femur adsorbed prior to fracture in the biomechanical evaluation. In some instances, QK-De10 is unable to improve the fracture callus strength for type 1 OI mice.

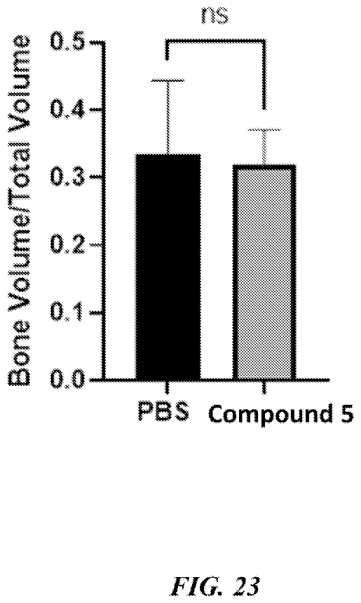

FIG. 23 shows the impact of treatment with the targeted compound QK-De10 on bone fracture callus mineralization in a Colla2^{oim} mouse model of type 1(+/−) OI. After reaching skeletal maturity 12 weeks old mice underwent a stabilized midshaft femur fracture model via an osteotomy. Type 1 OI mice were treated daily subcutaneously for 5 weeks post fracture. Fracture callus mineralization was evaluated via quantification of high resolution MicroCT(scanco) images of the fracture callus. In some instances, QK-De10 is unable to improve the fracture callus mineralization for type 1 OI mice.

In some instances, FIGS. 21-23 show that Compound 5 is unable to significantly improve bone strength and volume in mice with Type 1 OI. In some instances, FIGS. 21-23 show that a compound lacking direct osteoblast stimulatory activity is unable to improve bone strength and volume in mice with Type 1 OI. As suggested by the data provided herein, the amount of bone formed is correlated with the ability of a compound to activate of osteoblast activity on the bone, such as underscoring that bone anabolics that increase differentiation or activity (and/or prevent the apoptosis) of osteoblasts, such as abaloparatide, PTHrP, Ln2P3, or the like, may be more likely to provide effective treatments for fracture healing in OI than anabolics that do not, such as QK. Likewise, anabolics that directly affect the production and quality of collagen may be more likely to provide effective treatments for fracture healing in OI than anabolics that do not.

Provided in certain embodiments herein is a compound, or a pharmaceutically acceptable salt thereof, having a structure XYZ, in which X is a bone anabolic agent, Y is a linker, and Z is a bone targeting ligand.

In some embodiments, X is a bone anabolic agent (e.g., an agent having bone anabolic activity).

In some embodiments, X is a bone anabolic agent that (directly) stimulates or activates osteoblasts. In some embodiments, X is an agent that increases the differentiation of osteoblasts. In some embodiments, X is an agent that increases the activity of osteoblasts. In some embodiments, X is an agent that prevents apoptosis of osteoblasts.

In some embodiments, X is a bone anabolic agent that (directly) affects the production of collagen. In some embodiments, X is an agent that increases collagen production.

In some embodiments, X is a bone anabolic agent that increases the production of collagen in the individual or improves the ability of the individual to form or process collagen.

In some embodiments, X is a bone anabolic agent that targets low collagen sites, such as at the fracture site, outside of a fracture site, or in the absence of a fracture site.

In some embodiments, X is a growth factor, a small molecule, a peptide, a protein, a hormone, or a fragment thereof, such as when attached to or released from a compound described herein.

In some embodiments, X is an antibody.

In some embodiments, X is a bone anabolic agent selected from the group consisting of an agonist of parathyroid hormone receptor 1, a parathyroid hormone (PTH), a PTH-related protein (PTHrP), and abaloparatide.

In some embodiments, X is a bone anabolic agent (e.g., having bone anabolic activity) including growth factors, small molecules, peptides, proteins, or hormones. In some embodiments, In some embodiments, X is abaloparatide (e.g., or a derivative or fragment thereof (e.g., having bone anabolic activity)).

In some embodiments, X is an agonist of parathyroid hormone receptor 1.

In some embodiments, X is a PTH.

In some embodiments, X is a PTHrP.

In some embodiments, X is abaloparatide.

In some embodiments, X is Ln2P3.

In some embodiments, X is QK.

In some embodiments, Z is a hydroxyapatite targeting ligand.

In some embodiments, the hydroxyapatite targeting ligand includes a tetracycline, a phosphonate (e.g., a bisphosphonate (e.g., a mono-bisphosphonate, a tri-bisphosphonate, or a polybisphosphonate)), an acidic oligopeptide, a ranelate, a pyrophosphate, or a targeting ligand developed through phage display.

In some embodiments, the hydroxyapatite targeting ligand includes a tetracycline, a phosphonate (e.g., a bisphosphonate (e.g., a mono-bisphosphonate, a tri-bisphosphonate, or a polybisphosphonate)), an acidic oligopeptide, a ranelate, and/or a pyrophosphate.

In some embodiments, the hydroxyapatite targeting ligand comprises a phosphate to a phosphonate, or a derivative thereof.

In some embodiments, Z includes one or more amino acid residue. In some embodiments, Z is a linear chain of amino acid residues. In some embodiments, Z is a branched chain of amino acid residues.

In some embodiments, Z is an acidic oligopeptide. In some embodiments, Z includes at least 4 glutamic acid amino acid residues or 4 aspartic acid amino acid residues. In some embodiments, Z includes at least 4 amino acid residues. In some embodiments, Z includes at least 4 acidic amino acid residues. In some embodiments, Z includes at least 4 amino acid residues having the same chirality. In some embodiments, Z includes at least 4 amino acid residues, wherein one or more of the amino acid residues has D-chirality. In some embodiments, Z includes at least 4 amino acid residues, wherein one or more of the amino acid residues has D-chirality and another of the one or more of the amino acid residues has L-chirality. In some embodiments, Z includes at least 4 acidic amino acid residues having the same chirality. In some embodiments, each of the at least 4 amino acid residues has D chirality. In some embodiments, each of the at least 4 acidic amino acid residues has D chirality. In some embodiments, one or more amino acid residue has L chirality.

In some embodiments, Z includes at least 4 glutamic acid amino acid residues. In some embodiments, Z includes at least 4 D-glutamic acid amino acid residues. In some embodiments, Z includes 4 to 20 D-glutamic acid amino acid residues. In some embodiments, Z includes at least 4 aspartic acid amino acid residues. In some embodiments, Z includes at least 4 D-aspartic acid amino acid residues. In some embodiments, Z includes 4 to 20 D-aspartic acid amino acid residues. In some embodiments, Z includes at least 4 (e.g., D-) glutamic acid amino acid residues (e.g., 4 to 20 D-glutamic acid amino acid residues) and/or at least 4 (e.g., D-) aspartic acid amino acid residues (e.g., 4 to 20 D-aspartic acid amino acid residues). In some embodiments, Z includes a mixture of (e.g., D-) glutamic acid amino acid residues and (e.g., D-) aspartic acid amino acid residues.

In some embodiments, Z includes at least 10 repeating D-glutamic acid amino acid residues (e.g., DE10 or more, DE15 or more, or DE20 or more). In some embodiments, Z includes at least 15 repeating D-glutamic acid amino acid residues (e.g., DEI 5 or more or DE20 or more). In some embodiments, Z includes at least 20 repeating D-glutamic acid amino acid residues (e.g., DE20 or more). In some embodiments, Z is 20 repeating D-glutamic acid amino acid residues (DE20).

In some embodiments, Z includes 4 to 75 acidic amino acid residues (e.g., D-glutamic acid amino acid residues and/or D-aspartic acid amino acid residues). In some embodiments, Z includes 8 to 30 acidic amino acid residues (e.g., D-glutamic acid amino acid residues).

In some embodiments, Z includes 8 to 30 D-glutamic acid amino acid residues.

In some embodiments, Z includes 8 to 30 D-aspartic acid amino acid residues.

In some embodiments, Z is DE10.

In some embodiments, Z is DE20.

In some embodiments, X is abaloparatide (e.g., or a derivative or fragment thereof (e.g., having bone anabolic activity)) and Z is 20 repeating D-glutamic acid amino acid residues (DE20).

In some embodiments, Y is a non-releasable linker. In some embodiments, Y is a non-releasable linker containing at least one carbon-carbon bond. In some embodiments, Y is a non-releasable linker containing at least one amide bond. In some embodiments, Y is a non-releasable linker (e.g., containing at least one carbon-carbon bond and/or at least one amide bond).

In some embodiments, Y is a releasable linker. In some embodiments, Y is a releasable linker containing at least one disulfide (SS). In some embodiments, Y is a releasable linker containing at least one ester (e.g., O(C═O)). In some embodiments, Y is a releasable linker containing at least one amide bond. In some embodiments, Y is a releasable linker containing at least one protease-specific amide bond. In some embodiments, Y is a releasable linker (e.g., containing at least one disulfide (SS), at least one ester (e.g., O(C═O)), and/or at least one (e.g., protease-specific) amide bond.

In some embodiments, X is abaloparatide (e.g., or a derivative or fragment thereof (e.g., having bone anabolic activity)), Y is a non-releasable oligopeptide linker, and Z is 20 repeating D-glutamic acid amino acid residues (DE20).

In some embodiments, X is abaloparatide (e.g., or a derivative or fragment thereof (e.g., having bone anabolic activity)), Y is a releasable oligopeptide linker comprising at least one protease-specific amide bond, and Z is 20 repeating D-glutamic acid amino acid residues (DE20).

In some embodiments, the elements of XYZ may be those disclosed in U.S. Pat. No. 10,960,054 and U.S. patent application Ser. Nos. 17/058,884, 17/058,887, and 17/058,891, which are incorporated by reference in their entirety herein. In some embodiments, the elements of XYZ may be those disclosed Application Numbers PCT/US2017/064081, PCT/US2019/034759, PCT/US2019/034764, and PCT/US2019/034767, which are incorporated by reference in their entirety herein.

In some embodiments, the compound has at least 75% sequence identity or more (e.g., at least 80% sequence identify or more, at least 85% sequence identify or more, at least 90% sequence identify or more, or at least 95% sequence identify or more) to SEQ ID NO.: 1.

In some embodiments, X is PTHrP, Y is a non-releasable oligopeptide linker, and Z is 20 repeating D-glutamic acid amino acid residues (DE20).

In some embodiments, the compound has at least 75% sequence identity or more (e.g., at least 80% sequence identify or more, at least 85% sequence identify or more, at least 90% sequence identify or more, or at least 95% sequence identify or more) to SEQ ID NO.: 3.

In some embodiments, X is Ln2P3, Y is a non-releasable oligopeptide linker, and Z is 10 repeating D-glutamic acid amino acid residues (DE10).

In some embodiments, the compound has at least 75% sequence identity or more (e.g., at least 80% sequence identify or more, at least 85% sequence identify or more, at least 90% sequence identify or more, or at least 95% sequence identify or more) to SEQ ID NO.: 4.

In some embodiments, X is QK, Y is a non-releasable oligopeptide linker, and Z is 10 repeating D-glutamic acid amino acid residues (DE10).

In some embodiments, the compound has at least 75% sequence identity or more (e.g., at least 80% sequence identify or more, at least 85% sequence identify or more, at least 90% sequence identify or more, or at least 95% sequence identify or more) to Compound 5.

In some embodiments, a compound described herein, such as a compound for treating Osteogenesis Imperfecta (OI) in an individual (e.g., in need thereof), is Compound 1.

In some embodiments, a compound described herein, such as a compound for treating Osteogenesis Imperfecta (OI) in an individual (e.g., in need thereof), is Compound 3.

In some embodiments, a compound described herein, such as a compound for treating Osteogenesis Imperfecta (OI) in an individual (e.g., in need thereof), is Compound 4.

In some embodiments, X is PTH, Y is a non-releasable oligopeptide linker, and Z is 20 repeating D-glutamic acid amino acid residues (DE20).

### EXAMPLES

These examples are provided for illustrative purposes only and not to limit the scope of the claims provided herein.

### Example 1: In Vivo Osteogenesis Imperfecta Murine Studies

In vivo experiments were conducted in heterozygous and homozygous Colla2^{oim} (Type 1 OI (+/−) and Type 3 OI (−/−)) mice of both genders. After reaching skeletal maturity, 12 weeks old female and/or male mice underwent a stabilized midshaft femur fracture model via an osteotomy. OI mice were subcutaneously treated (with saline control or a compound provided herein, such as Ab46-D-Glu20) twice a week 5 or 6 weeks post fracture. Following a 5-week study in heterozygous mice and 6-week in homozygous mice, fracture callus mineralization/densities were measured using quantification of high resolution MicroCT(scanco) images of the fracture callus.

### Example 2: Biodistribution

To evaluate the ability of bone targeted therapeutics to deliver therapeutics to bone fracture surfaces, Colla2^{oim} type 3 mice (−/−) with and without fractures were injected with a compound provided herein (e.g., SEQ ID NO.: 2). The compound was injected into the mice to evaluate biodistribution. 21 hours post injection, the biodistribution of the compound was evaluated via SPEC-CT.

Specifically, SEQ ID NO.: 2 was injected in 12 week old female Colla2^{oim} type 3 mouse (−/−) mice with midshaft femur fractures 2 weeks post-fracture. The mice were imaged at 21 hours using SPECT/CT (MiLabs U-SPECT-IVCT). CT images were taken under high resolution full body 12-minute scans and were followed by 1-hour SPECT scans using a 0.3 mm collimator. SPECT images were reconstructed using the MiLabs software selecting the energy window of 140 keV and reconstruction parameters of 16 subsets and 4 iterations without post filter. 3D reconstructions were performed using Imaged software.

While preferred embodiments have been shown and described herein, it will be obvious to those skilled in the art that such embodiments are provided by way of example only. Numerous variations, changes, and substitutions will now occur to those skilled in the art without departing from this disclosure. It should be understood that various alternatives to the embodiments described herein might be employed in practicing current disclosure.

1. A method of treating Osteogenesis Imperfecta (OI) in an individual, the method comprising administering to the individual a therapeutically effective amount of a compound, or a pharmaceutically acceptable salt thereof, having a structure X-Y-Z, wherein X is a bone anabolic agent, Y is a linker, and Z is a bone targeting ligand.

2. (canceled)

3. The method of claim 1, wherein a therapeutically effective amount of the compound is delivered to one or more damaged, low density, or weakened bone sites.

4. The method of claim 1, wherein the compound comprises a diagnostic payload and administering the compound identifies the one or more damaged, low density, or weakened bone sites in the individual.

5. The method of claim 1, wherein the therapeutically effective amount of the compound, or the pharmaceutically acceptable salt thereof, reduces the incidences or likelihood of bone fracture in the individual.

6. The method of claim 1, wherein the therapeutically effective amount of the compound, or the pharmaceutically acceptable salt thereof, increases bone density or strength of a bone in the individual as compared to the bone density or strength of the bone prior to administration of the compound.

7. The method of claim 1, wherein the therapeutically effective amount of the compound, or the pharmaceutically acceptable salt thereof, accelerates the healing or repair of an osteotomy, a bone graft, or a bone fracture in the individual as compared to a healing or repair rate without administration of the compound.

8. The method of claim 1, wherein the OI is Type 1 OI, Type 3 OI, or Type 4 OI.

9-11. (canceled)

12. The method of claim 1, wherein the OI is pediatric OI.

13. The method of claim 1, wherein a therapeutically effective amount of the compound, or the pharmaceutically acceptable salt thereof, is delivered to one or more low density or weakened bone sites in the individual.

14-15. (canceled)

16. The method of claim 1, wherein X is a bone anabolic agent selected from the group consisting of an agonist of parathyroid hormone receptor 1, a parathyroid hormone (PTH), a PTH-related protein (PTHrP), and abaloparatide.

17. (canceled)

18. The method of claim 1, wherein Z is a hydroxyapatite targeting ligand.

19. The method of claim 1, wherein the hydroxyapatite targeting ligand comprises a tetracycline, a phosphate or a derivative thereof, a phosphonate (e.g., a bisphosphonate (e.g., a mono-bisphosphonate, a tri-bisphosphonate, or a polybisphosphonate)) or a derivative thereof, an acidic oligopeptide, a ranelate, a pyrophosphate, or a hydroxyapatite targeting ligand developed through phage display.

20. The method of claim 1, wherein Z is a linear chain of amino acid residues or a branched chain of amino acid residues.

21. The method of claim 1, wherein Z is a branched chain of amino acid residues.

22-23. (canceled)

24. The method of claim 1, wherein Z comprises at least 4 amino acid residues.

25. The method of claim 24, wherein each of the at least 4 amino acid residues has D chirality.

26. The method of claim 1, wherein Z comprises 10 to 20 D-glutamic acid amino acid residues.

27. The method of claim 1, wherein Z comprises a mixture of glutamic acid amino acid residues and aspartic acid amino acid residues.

28. (canceled)

29. The method of claim 1, wherein Z is 20 repeating D-glutamic acid amino acid residues (DE20).

30-32. (canceled)

33. The method of claim 1, wherein X is abaloparatide (or a derivative or fragment thereof having bone anabolic activity and Z is 20 repeating D-glutamic acid amino acid residues (DE20).

34. The method of claim 1, wherein Y is a non-releasable linker.

35. The method of claim 1, wherein Y is a releasable linker and/or at least one amide bond.

36. The method of claim 1, wherein X is abaloparatide, Y is a non-releasable oligopeptide linker, and Z is 20 repeating D-glutamic acid amino acid residues (DE20).

37. The method of claim 1, wherein X is abaloparatide, Y is a releasable oligopeptide linker comprising at least one protease-specific amide bond, and Z is 20 repeating D-glutamic acid amino acid residues (DE20).

38. The method of claim 1, wherein the compound has at least 75% sequence identity or more to SEQ ID NO: 1, SEQ ID NO: 3, or SEQ ID NO: 4.

39. (canceled)

40. The method of claim 1, wherein X is PTHrP.

41. The method of claim 1, wherein X is PTHrP, Y is a non-releasable oligopeptide linker, and Z is 20 repeating D-glutamic acid amino acid residues (DE20).

42-43. (canceled)

44. The method of claim 1, wherein X is Ln2P3.

45. The method of claim 1, wherein X is Ln2P3, Y is a non-releasable oligopeptide linker, and Z is 10 repeating D-glutamic acid amino acid residues (DE10).

46-47. (canceled)

48. A method of delivering a compound, or a pharmaceutically acceptable salt thereof, to one or more damaged, low density, or weakened bone sites in an individual having Osteogenesis Imperfecta (OI), the method comprising:
administering the compound to the individual, wherein the compound has a structure X-Y-Z, wherein:
X is a bone anabolic agent selected from the group consisting of an agonist of parathyroid hormone receptor 1, a parathyroid hormone (PTH), a PTH-related protein (PTHrP), and abaloparatide,
Y is a linker, and
Z is a bone targeting ligand comprising at least 4 amino acid residues having the same chirality.


SEQUENCE LISTING (4 sequences)

SEQ ID NO: 1 - protein, 65 aa, synthetic construct
  MOD_RES 28: note = X is Aib
  SITE 46..65: note = D-Glutamic acid
AVSEHQLLHDKGKSIQDLRRELLEKLLXKLHTAEIRATSEVSPNSEEEEEEEEEEEEEEEEEEEE

SEQ ID NO: 2 - protein, 25 aa, synthetic construct
  SITE 1..20: note = D-glutamic acid
  SITE 21: note = 2,2-amino ethoxy ethoxy acetic acid
  SITE 22: note = Beta diaminoproprionic acid
  SITE 25: note = chelated m99Tc
EEEEEEEEEEEEEEEEEEEEXXDCX

SEQ ID NO: 3 - protein, 67 aa, synthetic construct
  SITE 48..67: note = D-glutamic acid
AVSEHQLLHDKGKSIQDLRJTRFFLHHLIAEIHTAEIRATSEVSPNSEEEEEEEEEEEEEEEEEEEE

SEQ ID NO: 4 - protein, 23 aa, synthetic construct
  SITE 1..10: note = D-glutamic acid
  SITE 11: note = 2,2-amino ethoxy ethoxy acetic acid
  REPEAT 11: note = repeated 4 times
EEEEEEEEEEXDLTIDDSYWYRI